(19) United States
(12) Patent Application Publication
(10) Pub. No.: US 2004/0241109 A1
(43) Pub. Date: Dec. 2, 2004

(54) Multi-benefit oral hygiene product

(76) Inventor: Rita Parikh, Paramus, NJ (US)

Correspondence Address: PFIZER, INC., 201 TABOR ROAD, MORRIS PLAINS, NJ, 07950, US

(21) Appl. No.: 10/827,724

(22) Filed: Apr. 20, 2004

Related U.S. Application Data
(63) Continuation-in-part of application No. 10/369,947, filed on Feb. 18, 2003, now abandoned.
Application No. 10/369,947 is a continuation of application No. 10/049,155, filed on Feb. 5, 2002, now abandoned.
Application No. 10/049,155 is a 371 of international application No. PCT/US00/11398, filed on Apr. 28, 2000.
(60) Provisional application No. 60/140,599, filed on Jun. 23, 1999.

(51) Int. Cl. A61K 7/16; A61K 7/18

(52) U.S. Cl. 424/49

(57) ABSTRACT

The present invention is directed to an oral care composition including a synergistically effective combination of (i) at least one essential oil selected from the group consisting of thymol, menthol, eucalyptol, methyl salicylate, anethol and eugenol, and (ii) at least one lactic acid ingredient selected from the group consisting of lactic acid and salts of lactic acid. The pH of the composition is about 3.0 to about 5.5.

### BACKGROUND OF INVENTION

### FIELD OF THE INVENTION

The present invention relates to an antiseptic composition to help reduce oral malodor and caries, containing a synergistically effective combination of essential oils and lactic acid.

### BACKGROUND OF THE INVENTION

Volatile or essential oils are widely used in oral care products. Essential oils are aromatic compounds that are either derived from plant sources or are synthesized. Some essential oils show long-lasting germicidal effectiveness against the most common pathogens in the mouth. These pathogens are fkequeltly associated with oral malodor, plaque, and gingivitis. Thymol is an essential oil that is well-known and widely used as an antimicrobial in oral care products. Other essential oils include menthol, methyl salicylate, eucalyptol, anethol and eugenol.

Essential oils have been used for years ill antiseptic sold antiplaque mouthwash solutions. For example, LISTERINE® antiseptic mouthwash has been marketed since 1881, and contains the essential oils thymol, menthol, eucalyptol, and methyl salicylate.

More recently, essential oils have been included in formulations of toothpaste. For example. U.S. Pat. No. 1,526,940 to Staegemann teaches a toothpaste with the germicide ammonium ichthyol sulphonate with high amounts of thymol, menthol, eucalyptol, methyl salicylate, and peppermint oil as flavorants and taste-masking ingredients.

U.S. Pat. No. 3,164,524 to Fand et al. teaches an oral antiseptic comprising 2,2′-thiobis-(4,6-dichlorophenol), boric acid, methyl salicylate, thymol, menthol and eucalyptol.

U.S. Pat. No. 5,094,843 to Mazzanobile et al. teaches an anti-plaque, anti-gingivitis toothpaste with a fluorine source, and a specific range of thymol, menthol, methyl salicylate and eucalyptol.

European Patent Application 04974776 to Colgate-Palmolive Co. teaches an antiplaque oral composition, including a toothpaste, with triclosan. The antiplaque activity of the triclosan is increased by essential oils such as eucalyptol, thymol, methyl salicylate, and menthol.

U.S. patent application Ser. No. 08/280,098 discloses that the antiseptic activity of dentifrice compositions with essential oils is enhanced when the pH of the composition is between about 3.0 and about 5.5.

Lactic acid and salts thereof have also been employed in oral care products. See, e.g., Gijsen, “Lactic acid and the fight against tooth decay,” Manufacturing Chemist (December 1995), and the references cited therein, particularly, van der Hoeven, Caries Research, 23:146 (1989), Kashket, Arch Oral Biol., 37:187 (1992), Schaeken, Caries Research, 24:376 (1990), Schaeken, Caries Research, 27:277 (1993) and Graubner, Zahnarztliche Welt, 65:177 (1950).

According to an “Application Data” document printed in November of 1993 by Purac America, Inc., which manufactures lactic acid salts, a French patent identified as FR 3.610 M describes a toothpaste composition comprising 1.5 wt. % aluminum lactate. The Purac document cites WO 92/05765 and U.S. Pat. No. 4,647,452 as two other patent publications also disclosing toothpaste compositions comprising lactic acid salts.

WO 92/05765 at page 7, Example II, describes an anti-tartar toothpaste composition comprising 7 wt. % calcium lactate and 0.1 wt. % peppermint oil. Typically, a major proportion of peppermint oil consists of the essential oil menthol.

U.S. Pat. No. 4,647,452 describes toothpaste compositions comprising a salicylamide and zinc salt, such as dl-lactate. Unlike the instant invention, this patent requires salicylamide in the toothpaste composition to hinder the formation of tartar, halitosis and gingivitis.

U.S. Pat. No. 4,363,794 describes an oral composition for caries prophylaxis, comprising a stannous salt, a water-soluble fluoride salt and an orally acceptable acid, such as lactic acid. Peppermint oil and spearmint oil, which contain essential oils, are mentioned as suitable flavorings.

Although the prior art contemplates oral care products comprising lactic acid salts and oral care products comprising essential oils, it does not appear that the art teaches combining synergistically effective amounts of these ingredients to form a oral care product synergistically effective against dental caries and oral malodor.

It is therefore an object of the present invention to provide an antiseptic composition that helps reduce oral malodor, wherein the composition contains at least one essential oil and at least one lactic acid salt and/or lactic acid to synergistically enhance the antiseptic activity of the composition.

All references cited herein are incorporated herein by reference in their entireties.

### SUMMARY OF THE INVENTION

The present invention is directed to an oral care composition comprising a synergistically effective combination of (i) at least one essential oil selected from the group consisting of thymol, menthol, eucalyptol, methyl salicylate, anethol and eugenol, and (ii) at least one lactic acid ingredient selected from the group consisting of lactic acid and salts of lactic acid, wherein a pH of said composition is about 3.0 to about 5.5.

### DETAILED DESCRIPTION OF THE INVENTION

Oral care compositions according to the invention can be provided in a variety of forms, including liquids (e.g., a mouthwash), solids (e.g., a toothpowder), and semi-solids (e.g., a dental gel or a toothpaste). Most preferably, the composition is a dentifrice composition, which is defined as a composition that is to be used in conjunction with a toothbrush to clean accessible tooth surfaces.

A preferred embodiment of the invention is an improved stable acidic antiseptic and anticaries dentifrice composition comprising optimally adjusted concentrations of essential oils, at least one lactic acid salt and/or lactic acid, and at least one fluoride-releasing compound, combined with abrasives, gelling systems, ancillary flavor systems, and other additives formulated to have acceptable cosmetic properties at a pH of about 3.0 to about 5.5. Dentifrice compositions of this invention can also contain, but are not limited to, one or more of the following dentifrice additives: acidifiers, abrasives, surfactants, binders and thickeners, humectants, sweeteners, desensitizing agents, flavors, colors, and preservatives. The dentifrice composition of the invention is acidified to a pH of about 3.0 to about 5.5 by acidifiers including, but not limited to, lactic acid, phosphoric acid, acidic phosphate salts, benzoic acid, and food grade acids (e.g., citric acid). The preceding active ingredients and additives are combined in a hydrous or anhydrous vehicle to form a solid (e.g., a toothpowder), a semi-solid (e.g., a paste or a gel), or a liquid (e.g., a mouthwash).

The compositions of the invention comprise at least one essential oil and at least one lactic acid salt and/or lactic acid, preferably combined in synergistically effective amounts. Preferably, the compositions comprise at least two essential oils selected from the group consisting of thymol, menthol, eucalyptol, methyl salicylate, anethol and eugenol, more preferably at least three of said essential oils, and most preferably at least four of said essential oils.

Compositions of this invention contain essential oils that have antiseptic properties. Essential oils are volatile aromatic oils which may be synthetic or may be derived from plants by distillation, expression or extraction, and which usually carry the odor or flavor of the plant from which they are obtained. In the composition of this invention, antiseptic activity is provided by essential oils. Some of these essential oils also act as flavoring agents. The essential oils of this invention include but are not limited to thymol, menthol, methyl salicylate (wintergreen oil), eucalyptol, carvacrol, camphor, anethole, carvone, eugenol, isoeugenol, limonene, osimen, n-decyl alcohol, citronel, δ-salpineol, methyl acetate, citronellyl acetate, methyl eugenol, cineol, linalool, ethyl linalaol, safrola vanillin, spearmint oil, peppermint oil, lemon oil, orange oil, sage oil, rosemary oil, cinnamon oil, pimento oil, laurel oil, cedarleaf oil, and clove oil. Thymol is the-most preferred essential oil.

Essential oils may be in the composition of the present invention in an amount of from about 0.1% w/w to about 4.0% w/w; preferably in an amount of from about 0.5% w/w to about 3.0% w/w; and most preferably in an amount of from about 1.0% w/w to about 2.0% w/w.

Thymol, also known by the chemical formula 5-methyl 2-(1-methylethyl) phenol, is obtained from the essential oil of Thymus vulgaris Labiatae and Monarda punctata Labiatae. Thymol is a white crystalline powder with an aromatic odor and taste and is soluble in organic solvents but only slightly soluble in deionized water. Thymol may be in the composition of this invention in an amount of from about 0.01% w/w to about 1.0% w/w; preferably in an amount of from about 0.1% w/w to about 0.6% w/w; and most preferably in an amount of from about 0.2% w/w to about 0.5% w/w.

Menthol is isolated principally from the oil of Mentha arvensis. In its commercial form, menthol is available as L-menthol crystals obtained from a process involving cooling of the oil. Fractional distillation of peppermint oil which usually contains from about 40% to about 65% menthol represents another important source of menthol. Synthetic sources of L-menthol are also available. Menthol may be in the composition of the present invention in an amount of from about 0.01% w/w to about 1.0% w/w; preferably in an amount of from about 0.10% w/w to about 0.7% w/w; and most preferably in an amount of from about 0.1% w/w to about 0.6% w/w.

Eucalyptol, another essential oil with antiseptic properties, is derived from the eucalyptus tree. Having a camphoraceous odor and cooling taste, this essential oil is often combined with other essential oils such as menthol in confection, formulations to impart medicinal effect. Combinations of menthol and eucalyptol are widely used. Particularly preferred uses of the menthol-eucalyptol combination include, according to the present invention, oral care products, such as toothpastes or dental gels. Eucalyptol may be in the composition of the present invention in an amount of from about 0.01% w/w to about 1.0% w/w; preferably in an amount of from 0.05% w/w to about 0.5% w/w; and most preferably in an amount of from about 0.07% w/w to about 0.4% w/w.

Methyl salicylate is the main ingredient in many essential oils, constituting about 99% of oil of wintergreen (Gaultheria procumbens) and sweet birch (Betula lenta). Methyl salicylate, which has a distinctive refreshing aroma, is used widely in mouthwashes, chewing gums and other oral and pharmaceutical preparations. Methyl salicylate may be in the composition of the present invention in an-amount of from about 0.01% w/w to about 1.0% w/w; preferably in an amount of from about 0.04% w/w to about 0.6% w/w; and most preferably in an amount of from about 0.1% w/w to about 0.6% w/w.

The composition of the invention may contain the following essential oils in percentages by weight: (a) thymol from about 0.01% w/w to about 1.0% w/w; (b) menthol from about 0.01% w/w to about 1.0% w/w; (c) eucalyptol from about 0.01% w/w to about 1.0% w/w; and (d) methyl salicylate from about 0.01% w/w to about 1.0% w/w.

In the preferred embodiment of the composition of the present invention, the composition may contain the Following essential oils in percentages by weight: (a) thymol from about 0.1% w/w to about 0.6% w/w; (b) menthol from about 0.1% w/w to about 0.7% w/w; (c) eucalyptol from about 0.05% w/w to about 0.5% w/w; and (d) methyl salicylate from about 0.04% w/w to about 0.6% w/w.

In the most preferred embodiment of the composition of the present invention, the composition may contain the following essential oils in percentages by weight: (a) thymol from about 0.2% w/w to about 0.5% w/w; (b) menthol from about 0.1% w/w to about 0.6% w/w; (c) eucalyptol from about 0.07% w/w to about 0.4% w/w; and (d) methyl salicylate from about 0% w/w to about 0.6% w/w.

Lactic acid and/or salts thereof combine with the essential oils of the composition to synergistically improve the hygienic properties of the composition of the invention. L-(+)-lactic acid and salts thereof are preferred, with the aluminum, calcium and zinc salts of L-(+)-lactic acid being most preferred. Suitable lactic acid salts are available, for example, from Purac America, Inc. (Lincolnshire, Ill.) under the marks Puracal®, Puramex® Al and Puramex® Zn.

The lactic acid and/or salt thereof and the at least one essential oil are preferably added to the composition in amounts synergistically effective to hinder the growth of plaque, the growth of tartar, the growth of germs that cause bad breath and/or tooth decay.

The composition of the invention preferably contains the lactic acid ingredient(s) (i.e., at least one lactic acid salt and/or lactic acid) in a total concentration of about 0.10% w/w to about 15% w/w, more preferably about 1% w/w to about 10% w/w, most preferably about 3% w/w to about 7% w/w.

In certain embodiments, the composition of the invention further comprises at least one fluoride-releasing compound. The at least one fluoride-releasing compound can be fully or slightly water soluble, and is characterized by its ability to release fluoride ions or fluoride-containing ions in water and by its lack of reaction with other components in the composition. In the dentifrice composition of the present invention, anticaries activity is provided by fluoride-releasing compounds. Suitable fluoride-releasing compounds include inorganic fluoride salts such as water-soluble alkaline earth metal, alkali metal, and heavy metal salts. Sodium monofluorophosphate, sodium fluoride, stannous fluoride and mixtures thereof are preferred.

The amount of fluoride-releasing compound present in a preferred embodiment of this invention depends upon the type of fluoride-releasing compound employed, the solubility of the fluoride-releasing compound, and the formulation of the dentifrice composition. The fluoride-releasing compound used must be used in a nontoxic amount. In general, the fluoride-releasing compound, when used, will be present in an amount by weight of up to about 1.2% w/w, preferably from about 0.1% w/w to about 1.0% w/w, and most preferably from about 0.175% w/w to about 0.8% w/w, of the dentifrice composition so as to release 800-1500 ppm F⁻.

The most preferred fluoride-releasing compound in the dentifrice composition of the invention is sodium monofluorophosphate at a concentration from about 0.5% w/w to about 1.0% w/w, more preferably about 0.65% w/w to about 0.88% w/w, or most preferably, 0.76% w/w.

The composition has an acid pH of about 3.0 to about 5.5. In this pH range the antiseptic activity of the composition is enhanced. A pH greater than about 5.5 decreases the antiseptic activity of the dentifrice composition. A pH below about 3.0 generally irritates the oral cavity.

The pH of the claimed dentifrice is adjusted to below 5.5 using suitable food or pharmaceutical grade acidifiers, including lactic acid. These could include, but are not limited to one or a combination of the following: phosphoric acid, benzoic acid, citric acid, or other tricarboxylic acids, and the like. The most preferred acidifiers in the present invention include a mixture of phosphoric acid from about 0.01% w/w to about 3.0% w/w, preferably-in the range of from about 0.1% w/w to about 1.5% w/w, and most preferably in the range of from about 0.2% w/w to about 0.75% w/w; monobasic sodium phosphate from about 0.01% w/w to about 1% w/w, preferably from about 0.1% w/w to about 0.5% w/w, and most preferably from about 0.2% w/w to about 0.4% w/w; dibasic sodium phosphate from about 0.001% w/w to about 1.0% w/w, preferably from about 0.01% w/w to about 0.5% w/w, and most preferably from about 0.01% w/w to about 0.05% w/w; and benzoic acid in the range of from about 0.01% w/w to about 1.0% w/w, preferably from about 0.05% w/w to about 0.5% w/w, and most preferably from about 0.08% w/w to about 0.35% w/w. The exact amount of acidifier added will depend on the final pH and buffer capacity desired.

The pH of the products may be buffered with salts of the acids in question, including lactic acid salts. Common buffer systems include phosphoric acid and sodium phosphate salts, or citric acid and sodium citrate. Suitable buffers for use in this invention include citric acid-sodium citrate, phosphoric acid-sodium phosphate, sodium monobasic phosphate-sodium dibasic phosphate, acetic acid-sodium acetate, and benzoic acid and benzoate in amounts up to about 1% w/w, preferably from about 0.05% w/w to about 0.75% w/w of the composition, and most preferably from about 0.1% w/w to about 0.5% w/w of the composition.

The preferred embodiment of the present invention may also contain conventional dentifrice additives including but not limited to humectants, binders, thickeners, surfactants, preservatives, sweeteners, flavors, colors, glycerin, and a buffer. These additives are present in amounts that do not interfere with the antiseptic and anticaries properties of the composition of the present invention.

Surfactants or surface active agents are organic compounds which reduce surface tension between liquids and aid in the dispersion of a composition throughout the oral cavity. The surfactant in the present invention may be anionic, nonionic, or amphoteric. The oral hygiene or dentifrice compositions of lo the present invention may contain surfactants in amounts up to about 5.0% w/w; preferably from about 0.1% w/w to about 3.0% w/w of the dentifrice composition; and most preferably from about 0.2% w/w to about 2.0% w/w of the dentifrice composition.

The most preferred surfactants are anionic. These anionic surfactants include but are not limited to sodium lauryl sulfate, sodium lauroyl sarcosinate, sodium methyl cocoyl taurate, and disodium lauryl sulfosuccinate.

In the most preferred embodiment the surfactant is the anionic surfactant sodium lauryl sulfate.

Amphoteric surfactants have the capacity to behave as either an acid or a base and include quaternized imidazole derivatives useful in the present invention. Preferred amphoteric surfactants include long chain (alkyl) amino-alkylene aklylated amine derivatives, also known as MIRANOL®, manufactured by Rhone-Poulanc, Cranberry, N.J.

Sweeteners well known in the art, including natural and artificial sweeteners, may be used. The sweetener may be selected from a wide range of materials including naturally occurring water-soluble sweeteners, artificial water-soluble sweeteners and modified water-soluble sweeteners derived from naturally occurring water-soluble sweeteners. Artificial water-soluble sweeteners include, but are not limited to, soluble saccharin salts, e.g., sodium or calcium saccharin salts, cyclamate salts, the sodium, ammonium or calcium salt of 3,4-dihydro-6-methyl-1,2,3-oxathia-zine-4-one-2,2-dioxide, the potassium salt of 3,4-dihydro-6-methyl-1,2,3-oxathia-zine-4-one-2,2-dioxide (Acesulfame-K), the free acid form of saccharin, and the like; dipeptide based sweeteners, such as L-aspartic acid derived sweeteners, e.g., L-aspartyl-L-phenylalanine methyl ester (Aspartame) and materials described in U.S. Pat. No. 3,492,131, L-alpha-aspartyl-N-(2,2,4,4-tetramethyl-3-thietanyl)-D-alaninamide hydrate (Alitame), methyl esters of L-aspartyl-L-phenylglycerine, and L-aspartyl-L-2,5-dihydrophenyiglycine, L-aspartyl-2,5-dihydro-L-phenylalanine, L-aspartyl-L-(1-cyclohexene)-alanine, and the like. Naturally occurring water-soluble sweeteners include, but are not limited to, sugar alcohols, including sorbitol as 70% sorbitol solution, mannitol, xylitol, maltitol, hydrogenated starch hydrolysates and mixtures thereof.

Water-soluble sweeteners derived from naturally occurring water-soluble sweeteners include, but are not limited to, chlorinated derivatives of sucrose, known, for example, under the product designation of Sucralose; and protein-based sweeteners such as thaumaoccous danielli (Thaumatin I and II).

Sorbitol solution supplies sweetness and body to the composition and gives a desirable mouth feel. Sorbitol solution also enhances flavor, prevents harsh taste and provides a fresh and lively sensation in the mouth. It also prevents caking of the dentifrice.

In general, an effective amount of sweetener is utilized to provide the level of sweetness desired in any particular embodiment of the dentifrice compositions according to the present invention. This amount will vary with the sweetener selected and the final oral hygiene product. The amount of sweetener normally present is from about 0.0025% w/w to about 60% w/w of the dentifrice composition. The exact range of amounts for each type of sweetener in a dentifrice is well known in the art and is not the subject of the present invention.

The flavors which may be used in the invention include natural and artificial flavors known in the dentifrice art. Suitable flavors include, but are not limited to, mints, such as peppermint, citrus flavors such as orange and lemon, artificial vanilla, cinnamon, various fruit flavors, and the like. Anethole (or anise camphor, p-propenyl anisole) is a flavor constituent of anise and fennel oils which are used widely as flavoring agents and antiseptics and was found useful in masking the harsh taste of thymol.

The amount of flavor is normally a matter of preference subject to such factors as the type of final dentifrice composition, the individual flavor employed, and the strength of flavor desired. The flavors are preferably utilized in amounts that may range in total amounts from about 0.01% w/w to about 6% w/w of the dentifrice composition.

Coloring agents in this invention are used in amounts effective to produce a dentifrice of the desired color. These coloring agents may be incorporated in amounts up to about 3% by weight of the dentifrice composition of the present invention. The coloring agents may also include natural food colors and dyes suitable for food, drug and cosmetic applications. These coloring gents are known as FD & C dyes and lakes. The materials acceptable for the foregoing uses are preferably water-soluble. Illustrative nonlimiting examples include the indigoid dye known as FD & C Blue No.1, and D & C Yellow No. 10. A full recitation of all FD & C colorants and their corresponding chemical structures may be found in the Kirk-Othmer Encyclopedia of Chemical Technology, 3rd Edition, in volume 5 at pages 857-884. A preferred opacifier, titanium dioxide, may be incorporated in amounts up to about 2.0% w/w, preferably less than about 1.0% w/w of the composition, and most preferably less than about 0.4% w/w.

Suitable humectants in this invention include sorbitol, as 70% sorbitol solution, glycerin, propylene glycol, polyethylene glycol, mixtures thereof, and the like. Humectants may be present in amounts from about 1.0% to about 75.0% by weight of the dentifrice composition.

Suitable abrasive substances for use in this invention include hydrated silica, calcium carbonate, calcium pyrophosphate, dicalcium phosphate dihydrate, or alkali metal meta-phosphates. Silica abrasives in the dentifrice composition according to this invention may include among others, ZEODENT® (113), manufactured by J. M. Huber Corp. and SYLOID® or SYLODENT®, manufactured by W.R. Grace Co. These polishing agents may be used in amounts up to about 75.0% w/w of the composition, preferably in amounts from about 5.0% w/w to about 40% w/w of the composition, and most preferably from about 5.0% w/w to about 30.0% w/w of the composition.

In the preferred embodiment of this invention, the composition includes an oral vehicle and is a dentifrice, such as a toothpaste or a dental gel. The dentifrice composition of this invention may also include binders or gelling agents to give the products their characteristic consistency. Gelling agents such as hydroxyethyl cellulose, carboxymethyl cellulose, methyl cellulose, xanthan gum, gelling silicas, and the like may be used singly or in combination. The preferred gelling system is a mixture of carboxy methyl cellulose, xanthan gum and gelling silica. Gelling agents may be used in amounts from about 0.5% w/w to about 30% w/w, preferably from about 5.0% w/w to about 15.0% w/w of the dentifrice composition, and most preferably from about 7.0% w/w to about 20% w/w of the composition.

The composition of this invention may also contain a desensitizing agent such as strontium chloride, potassium nitrate or sodium citrate-citric acid, which may be used in an amount from about 0.5% w/w to about 10% w/w.

Suitable preservatives in this invention include benzoic acid, butylated hydroxyanisole (BHA), butylated hydroxntoluene (BHT), ascorbic acid, methyl paraben, propyl paraben, tocopherols and mixtures thereof. Preservatives when used are generally present in amounts up to about 1.0% w/w, and preferably from about 0.1 % w/w to about 1.0% w/w of the dental gel composition.

The present invention extends to methods of makings the improved oral antiseptic compositions. In such a method, an oral antiseptic dentifrice composition according to the present invention is made by first combining water, part of the humectant, one or more sweeteners, phosphate salts and benzoic acid. If fluoride-releasing compounds are used, they are added in this step. The remainder of the humectant is separately combined with one or more gums, and then combined with the first mixture. Titanium dioxide and silicas are separately mixed, and combined with the other mixture previously prepared. Finally, colors, flavors, and surfactants are added and mixed. The pH is adjusted, as needed, with acidifiers. A vacuum is pulled if necessary for deaeration. The pH of a 25.0% w/w aqueous solution of the composition is measured using a suitable pH meter (e.g., Orion Research Microprocessor pH/millivolt Meter, Model 811).

The apparatus useful in accordance with the present invention comprises mixing apparatus well known in the dental art, and therefore the selection of the specific apparatus will be apparent to the artisan.

The present invention is further illustrated by the following prospective examples which are, however, not included to limit the effective scope of the claims. All parts and percentages in the examples and throughout the specification and claims are by weight of the final composition unless otherwise specified.

### EXAMPLES

### Assay for Dentifrice Antiseptic Activity

The relative antiseptic activity of the dentifrice compositions of the invention and other dentifrice compositions are tested using a kinetic kill time assay of bacterial suspensions, in particular Pseudomonas aeruginosa ATCC strain 10145 as the preferred test organism. According to this assay, bacterial suspensions containing approximately 10⁸-10⁹ organisms per ml are vigorously mixed with 25% slurries of dentifrice (1 part dentifrice: 3 parts diluent, w/v as described below) in a 1:9 ratio. Ten 1L aliquots of the assay mixture are removed with sterile bacteriological loops and streaked on nutrient agar at 30, 60, and 120 seconds, which periods are presumed to represent the range of time typically spent in toothbrushing. In Experimental Example 1, water is used as the dentifrice diluent. In Experimental Examples 2 & 3, 0.05 M phosphate buffered saline is used as a diluent. Relative antiseptic activity of the dentifrice is rated as a function of the number of surviving organisms remaining over time. When there are fewer than 100-150 colonies, actual counts are made; denser growth is estimated in three categories, based on a certain colony density, as e.g., 150-300, 300-700, and confluent.

### Experimental Example 1

Dentifrice formulations of this invention are prepared using ingredients and quantities as shown in Table 1. Comparative Formula A is made according to the example in the specification of U.S. Pat. No. 5,094,843 to Mazzanobile et al. Comparative Formula B is identical to Comparative Formula A except that Comparative Formula B is acidified to a pH of 4.5 with phosphoric acid. Formulas 1.1-1.3 of this invention are formulated with increasing levels of the essential oils thymol, menthol, methyl salicylate and eucalyptol. Formula 1.4 of this invention contains only thymol, at the concentration contained in Formula

Table 2 shows prospective results from antiseptic activity-assays run on the formulations in Table 1. The data demonstrate the following:

Comparative Formula A has a pH of 6.67, and exhibits no detectable antiseptic activity. Comparative Formula B, identical to Comparative Formula A except that it is acidified to a pH of 4.5, shows markedly increased antiseptic activity compared to Comparative Formula A. These data show the critical effect that pH has on antiseptic activity of dentifrice compositions containing essential oils. The antiseptic activity of Comparative Formula B is not as high as that of Inventive Formulas 1.1-1.4. Since the level of essential oils in Comparative Formula B and Inventive Formula 1.1 are similar, the improved antiseptic activity of Inventive Formula 1.1 suggests that other ingredients in Inventive Formula 1.1 optimize the antiseptic activity of the essential oils. The high level of antiseptic activity of Formulas 1.1-1.4 indicates that this activity occurs over a range of essential oil concentrations and combinations, providing that the pH of the composition is maintained at about 4.5.

TABLE 1
Formulations of Experimental Example 1

| | FORMULA NUMBER | | | | | |
|---|---|---|---|---|---|---|
| | A | B | 1.1 | 1.2 | 1.3 | 1.4 |
| PH | 6.67 | 4.5 | 4.46 | 4.46 | 4.51 | 4.51 |
| THYMOL | 0.291 | 0.291 | 0.310 | 0.479 | 0.639 | 0.479 |
| METHYL | 0.324 | 0.324 | 0.300 | 0.450 | 0.600 | 0 |
| SALICYLATE | | | | | | |
| MENTHOL | 0.226 | 0.226 | 0.210 | 0.318 | 0.425 | 0 |
| EUCA- | 0.389 | 0.389 | 0.460 | 0.691 | 0.922 | 0 |
| LYPTOL | | | | | | |
| LACTIC ACID | 0 | 0 | 3.000 | 0 | 0 | 0 |
| COPPER | 0 | 0 | 0 | 5.000 | 0 | 0 |
| LACTATE | | | | | | |
| Al LACTATE | 0 | 0 | 0 | 0 | 5.000 | 0 |
| ZINC | 0 | 0 | 0 | 0 | 0 | 5.000 |
| LACTATE | | | | | | |
| GLYCERINE | 10.000 | 10.000 | 6.000 | 6.000 | 6.000 | 6.000 |
| SORBITOL | 36.640 | 36.640 | 32.000 | 32.000 | 32.000 | 32.000 |
| SOLUTION | | | | | | |
| (70%) | | | | | | |
| WATER | 21.339 | 19.839 | 33.580 | 30.922 | 30.274 | 31.423 |
| PEG 400 | 3.000 | 3.000 | 0 | 0 | 0 | 0 |
| PROPYLENE | 0 | 0 | 0 | 0 | 0 | 0.958 |
| GLYCOL | | | | | | |
| XANTHAN | 0.900 | 0.900 | 1.000 | 1.000 | 1.000 | 1.000 |
| GUM | | | | | | |
| Na CMC | 0 | 0 | 0.500 | 0.500 | 0.500 | 0.500 |
| 12M31P | | | | | | |
| Na₂PO₃F | 0 | 0 | 0.760 | 0.760 | 0.760 | 0.760 |
| (MFP) | | | | | | |
| SODIUM | 0.221 | 0.221 | 0 | 0 | 0 | 0 |
| FLUORIDE | | | | | | |
| Na | 0.214 | 0.214 | 0.350 | 0.350 | 0.350 | 0.350 |
| SACCHARIN | | | | | | |
| NaH₂PO₄ | 0 | 0 | 0.250 | 0.250 | 0.250 | 0.250 |
| (monobasic) | | | | | | |
| Na₂HP0₄ | 0 | 0 | 0.030 | 0.030 | 0.030 | 0.030 |
| (dibasic) | | | | | | |
| BENZOIC | 0 | 0 | 0.250 | 0.250 | 0.250 | 0.250 |
| ACID | | | | | | |
| SODIUM | 0.200 | 0.200 | 0 0 | 0 | 0 | 0 |
| BENZOATE | | | | | | |
| Ti02 | 0.956 | 0.956 | 1.000 | 1.000 | 1.000 | 1.000 |
| GELLING | 10.000 | 10.000 | 6.000 | 6.000 | 6.000 | 6.000 |
| SILICA | | | | | | |
| ABRASIVE | 4.000 | 14.000 | 11.000 | 11.000 | 11.000 | 11.000 |
| PHOSPHORIC | 0 | 1.500 | 1.500 | 1.500 | 1.500 | 1.500 |
| ACID | | | | | | |
| (25% Aq.) | | | | | | |
| SLS | 1.300 | 1.300 | 1.500 | 1.500 | 1.500 | 1.500 |

TABLE 2
Antiseptic Activity of Table 1 Formulas

| | | BACTERIAL RECOVERY* | | |
|---|---|---|---|---|
| FORMULA # | pH | 0.5 MIN | 1 MIN | 2 MIN |
| A | 6.67 | CONFLUENT | CONFLUENT | CONFLUENT |
| B | 4.5 | CONFLUENT | [symbol] 500 | 1 |
| 1.1 | 4.46 | <22 | 0 | 0 |
| 1.2 | 4.46 | 0 | 0 | 0 |
| 1.3 | 4.51 | 0 | 0 | 0 |
| 1.4 | 4.51 | <2 | <1 | <1 |

*Colony-forming units surviving

### Experimental Example 2

A split factorial designed study is performed in order to examine the relation between dentifrice pH, abrasive levels, and antiseptic activity of dentifrice compositions using the inventive formulations in Table 3. In this study, the pH of the formulations ranges from 3.7 to 5.3. SYLODENT® 750 silica abrasive, available from W.R. Grace Co. is added at levels of 9% and 13% in the compositions formulated at pH 4.4-4.9. Compositions at all other pH levels are formulated with 11% abrasive. The data shown in Table 4 indicate that dentifrice compositions formulated at pH levels of 4.5 or below exhibit similar, high levels of antiseptic activity. The two compositions formulated at pH 4.9 also exhibit significant antiseptic activity, but to a somewhat lesser extent than the products with lower pH. The dentifrice formulated at pH 5.3 indicates significantly less antiseptic activity than the lower pH products. This provides additional evidence of the strong relationship between lower pH and increased antiseptic activity of dentifrices containing essential oils. While the products formulated with 13% silica abrasive demonstrate a trend toward slightly lower levels of antiseptic activity than those formulated with 9% abrasive at the same pH level, the difference is trivial. This indicates that varying abrasive levels between 9% and 13% has no significant impact on antiseptic activity.

TABLE 3
Antiseptic Dentifrice Formulas - pH & Abrasive Variants

| | FORMULA NO. | | | | | | |
|---|---|---|---|---|---|---|---|
| | 3.1 | 3.2 | 3.3 | 3.4 | 3.5 | 3.6 | 3.7 |
| pH | 3.7 | 4.09 | 4.12 | 4.5 | 4.9 | 4.9 | 5.29 |
| THYMOL | 0.479 | 0.479 | 0.479 | 0.479 | 0.479 | 0.479 | 0.479 |
| METHYL | 0.058 | 0.058 | 0.058 | 0.058 | 0.058 | 0.058 | 0.058 |
| SALICYLATE | | | | | | | |
| MENTHOL | 0.319 | 0.319 | 0.319 | 0.319 | 0.319 | 0.319 | 0.319 |
| EUCALYPTOL | 0.175 | 0.175 | 0.175 | 0.175 | 0.175 | 0.175 | 0.175 |
| PEPPERMINTI | 0.416 | 0.416 | 0.416 | 0.416 | 0.416 | 0.416 | 0.416 |
| SPEARMINT | | | | | | | |
| BLEND | | | | | | | |
| CALCIUM | 5.000 | 6.000 | 7.000 | 5.000 | 6.000 | 7.000 | 5.000 |
| LACTATE | | | | | | | |
| GLYCERINE | 6.000 | 6.000 | 6.000 | 6.000 | 6.000 | 6.000 | 6.000 |
| SORBITOL (70%) | 32.000 | 32.000 | 32.000 | 32.000 | 32.000 | 32.000 | 32.000 |
| WATER, | 25.713 | 23.663 | 26.613 | 27.613 | 25.263 | 26.923 | 28.863 |
| DEIONIZED | | | | | | | |
| XANTHAN GUM | 1.000 | 1.000 | 1.000 | 1.000 | 1.000 | 1.000 | 1.000 |
| Na CMC 12M31P | 0.400 | 0.400 | 0.400 | 0.400 | 0.400 | 0.400 | 0.400 |
| SODIUM CMC | 0.400 | 0.400 | 0.400 | 0.400 | 0.400 | 0.400 | 0.400 |
| 7MF | | | | | | | |
| Na F PO₄ (MFP) | 0.760 | 0.760 | 0.760 | 0.760 | 0.760 | 0.760 | 0.760 |
| Na SACCHARIN | 0.800 | 0.800 | 0.800 | 0.800 | 0.800 | 0.800 | 0.800 |
| PHOSPHORIC | 3.300 | 2.350 | 2.400 | 1.400 | 0.750 | 2.090 | 0.150 |
| ACID (25% Aq.) | | | | | | | |
| NaH₂PO₄ | 0.250 | 0.250 | 0.250 | 0.250 | 0.250 | 0.250 | 0.250 |
| (monobasic) | | | | | | | |
| Na₂HPO₄ | 0.030 | 0.030 | 0.030 | 0.030 | 0.030 | 0.030 | 0.030 |
| (dibasic) | | | | | | | |
| TiO2 | 1.000 | 1.000 | 1.000 | 1.000 | 1.000 | 1.000 | 1.000 |
| GELLING SILICA | 7.000 | 7.000 | 7.000 | 7.000 | 7.000 | 7.000 | 7.000 |
| ABRASIVE | 11.000 | 13.000 | 9.000 | 11.000 | 13.000 | 9.000 | 11.000 |
| SILICA | | | | | | | |
| BENZOIC ACID | 0.150 | 0.150 | 0.150 | 0.150 | 0.150 | 0.150 | 0.150 |
| SLS | 1.500 | 1.500 | 1.500 | 1.500 | 1.500 | 1.500 | 1.500 |
| FD&C BLUE 1 | 2.000 | 2.000 | 2.000 | 2.000 | 2.000 | 2.000 | 2.000 |
| (0.1% Aq.) | | | | | | | |
| D&C YELLOW 10 | 0.250 | 0.250 | 0.250 | 0.250 | 0.250 | 0.250 | 0.250 |
| (0.1.% Aq.) | | | | | | | |

TABLE 4
Antiseptic Activity of Table 3 Formulas

| | | BACTERIAL RECOVERY* | | |
|---|---|---|---|---|
| | | AFTER EXPOSURE | | |
| FORMULA # | pH | 0.5 MIN | 1 MIN | 2 MIN |
| 3.1 | 3.7 | <99 | <3 | <2 |
| 3.2 | 4.1 | <22 | <1 | 0 |
| 3.3 | 4.1 | <9 | <1 | <1 |
| 3.4 | 4.5 | <120 | <10 | <8 |
| 3.5 | 4.9 | <144 | <44 | <34 |
| 3.6 | 4.9 | <135 | <38 | <25 |
| 3.7 | 5.3 | <500 | <200 | <200 |

*Colony forming units (CFU) per time period. Data represent, means of duplicate assays conducted on two different days (i.e., N = 4). Confluent growth represents >500 CFU, with a theoretical limit of 10⁵.

### Experimental Example 3

Further evidence that the antiseptic activity of the invention is enhanced at low pH is provided by the comparative experiment summarized in Table 5. In this study the antiseptic activities of Inventive Formulas 1.3 and 1.4 are compared to certain commercial dentifrices having pH levels between 6.8 and 4.45. These commercial dentifrices do not contain thymol or antiseptically effective levels of other essential oils. The comparative formulations are formulated at acidic pH ranges to preserve the stability of their fluoride source or other active ingredients. The clear superiority of the inventive formulations is evidence of the importance of the antiseptic essential oils, lactic acid/lactic acid salts and low pH on antiseptic activity.

TABLE 5
Antiseptic Activity of Essential Oil Dentifrices Versus Other Low
pH Prior Art Formulations

| | | BACTERIAL RECOVERY* | | |
|---|---|---|---|---|
| | | AFTER EXPOSURE OF | | |
| FORMULA | pH | 0.5 MIN | 1 MIN | 2 MIN |
| COMPARATIVE | 6.8 | CONFLUENT | CON- | CON- |
| FORMULATION C | | | FLUENT | FLUENT |
| COMPARATIVE | 4.5 | CONFLUENT | >500 | >>250 |
| FORMULATION D | | | | |
| COMPARATIVE | 4.45 | CONFLUENT | >>200 | 0 |
| FORMULATION E | | | | |
| COMPARATIVE | 5.36 | CONFLUENT | >500 | >500 |
| FORMULATION F | | | | |
| COMPARATIVE | 5.69 | CONFLUENT | >500 | >>300 |
| FORMULATION G | | | | |
| COMPARATIVE | 5.69 | >>200 | 1 | 0 |
| FORMULATION H | | | | |
| INVENTIVE | 4.46 | <1 | 0 | 0 |
| FORMULA 1.3 | | | | |
| INVENTIVE | 4.46 | 0 | 0 | 0 |
| FORMULA 1.4 | | | | |

*Colony forming units per time period. Data represent means of duplicate assays conducted on two different days (i.e., N = 4). Confluent growth represents >500 CFU, with a theoretical limit of 10⁵.

### Experimental Example 4

A human oral malodor study is conducted in which a 6 member panel of trained hedonic judges evaluate the breath of 23 human subjects before and up to 4 hours after brushing with Inventive Formulas 1.3 and 1.5, and a placebo control consisting of the unflavored dentifrice base. The study employs a three-way crossover design in which each subject brushes with each product, with at least one day between evaluations. The panel of hedonic judges score the subjects blindly at baseline, and 30, 60, 90, 120, 180 and 240 minutes after brushing using a 9 point scale. The results indicate that brushing with the inventive formulas significantly reduces oral malodor scores relative to placebo at all post-brushing evaluations. Inventive Formula 1.5 reduces malodor somewhat better than Inventive Formula 1.3, suggesting a dose response to the antiseptic flavoring system.

### Example 5

One kilogram of a preferred embodiment of the invention is prepared with the ingredients shown in Table 6, using mixing and blending techniques known to those skilled in the art. The pH is adjusted with phosphoric acid to a pH of 4.3 to 4.7. The antiseptic activity of this formulation is comparable to that of Inventive Formulas 1.2 to 1.4. The dentifrice composition prepared in accordance with the Example exhibits excellent cosmetic properties including acceptable appearance, odor, and taste.

TABLE 6

| Ingredient | Percent W/W |
|---|---|
| Glycerin USP Special | 6.000 |
| Xanthan Gum K6B166 | 1.0000 |
| Sodium Carboxymethyl Cellulose Type 12M31 | .6000 |
| Sodium Carboxymethyl Cellulose USP Type | .6000 |
| Sorbitol Solution USP | 32.0000 |
| Sodium Monofluorophosphate 100% | .7600 |
| Saccharin Sodium (Spray Dried, FCC) | 1.2000 |
| Sodium Phosphate (Monobasic) Anhydrous | .2500 |
| Sodium Phosphate Anhydrous (Dibasic) | .0300 |
| Acid Benzoic USP | .1500 |
| Titanium Dioxide USP | .3500 |
| Hydrated Silicon Dioxide | 7.0000 |
| Silica Amorphous, Synthetic (Sylodent 750) | 11.0000 |
| Sodium Lauryl Sulfate, Washed & Dried | 1.5000 |
| Calcium Lactate | 7.0000 |
| Water Deionized | 28.5914 |
| Thymol NF | .4000 |
| Menthol USP | .1750 |
| Methyl Salicylate NF | .0600 |
| Eucalyptol | .1000 |
| Spearmint Oil Blend, Redistilled | .1000 |
| Peppermint Oil NF/FCC, Crystal White | .2000 |
| Mint Flavor N & A 42603 | .5200 |
| FD and C Blue No. 1 | .0010 |
| D and C Yellow No. 10 | .0001 |
| Phosphoric Acid NF | .4125 |
| Totals | 100.0000 |

While the invention has been described in detail and with reference to specific examples thereof, it will be apparent to one skilled in the art that various changes and modifications can be made therein without departing from the spirit and scope thereof.

What is claimed is:

1. An oral care composition comprising a synergistically effective combination of:
at least one essential oil selected from the group consisting of thymol, menthol, eucalyptol, methyl salicylate, anethol and eugenol, and
at least one lactic acid ingredient selected from the group consisting of lactic acid and salts of lactic acid,
wherein a pH of said composition is about 3.0 to about 5.5.

2. The composition of claim 1, wherein said combination is synergistically effective to hinder plaque growth, hinder tartar growth, kill germs and/or hinder tooth decay.

3. The composition of claim 2, wherein a total concentration of said at least one essential oil is about 0.1% w/w to about 4.0% w/w.

4. The composition of claim 3, wherein the total concentration of said is at least one essential oil is about 0.5% w/w to about 3.0% w/w.

5. The composition of claim 4, wherein the total concentration of said at least one essential oil is about 1.0% w/w to about 2.0% w/w.

6. The composition of claim 2, wherein a total concentration of said at least one lactic acid ingredient is about 0.10% w/w to about 15% w/w.

7. The composition of claim 6, wherein the total concentration of said at least one lactic acid ingredient is about 1% w/w to about 10% w/w.

8. The composition of claim 7, wherein the total concentration of said at least one lactic acid ingredient is about 3% w/w to about 7% w/w.

9. The composition of claim 6, wherein a total concentration of said at least one essential oil is about 0.1% w/w to about 4.0% w/w.

10. The composition of claim 7, wherein a total concentration of said at least one essential oil is about 0.5% w/w to about 3.0% w/w.

11. The composition of claim 8, wherein the total concentration of said at least one essential oil is about 1.0% w/w to about 2.0% w/w.

12. The composition of claim 9, wherein thymol is present in an amount of about 0.01% w/w to about 1.0% w/w, menthol is present in an amount of about 0.01% w/w to about 1.0% w/w, eucalyptol is present in an amount: of about 0.01% w/w to about 1.0% w/w, and methyl salicylate is present in an amount of about 0.01% w/w to about 1.0% w/w.

13. The composition of claim 10, wherein thymol is present in an amount of about 0.1% w/w to about 0.6% w/w, menthol is present in an amount of about 0.1% w/w to about 0.7% w/w, eucalyptol is present in an amount of about 0.05% w/w to about 0.5% w/w, and methyl salicylate is present in an amount of about 0.04% w/w to about 0.6% w/w.

14. The composition of claim 13, wherein thymol is present in an amount of about 0.2% w/w to about 0.5% w/w, menthol is present in an amount of about 0.1% w/w to about 0.6% w/w, eucalyptol is present in an amount of about 0.07% W/W to about 0.4% w/w, and methyl salicylate is present in an amount of about 0.1% w/w to about 0.6% w/w.

15. The composition of claim 2 in a form of a dentifrice further comprising at least one fluoride-releasing compound.

16. The composition of claim 15, wherein said at least one fluoride-releasing compound is selected from the group consisting of monofluophosphate, alkali metal fluoride, stannous fluoride, aluminum monofluorophosphate, aluminum difluorophosphate, and mixtures thereof.

17. The composition of claim 15, wherein a concentration of said fluoride-releasing compound is about 0.1% w/w to about 1.2% w/w.

18. The composition of claim 2, wherein said at least one lactic acid ingredient is an L-(+)-lactic acid salt selected from the group consisting of aluminum lactate, calcium lactate and zinc lactate.

19. The composition of claim 6, wherein said at least one lactic acid ingredient is an L-(+)-lactic acid salt selected from the group consisting of aluminum lactate, calcium lactate and zinc lactate.

20. The composition of claim 9, wherein said at least one lactic acid ingredient is an L-(+)-lactic acid salt selected from the group consisting of aluminum lactate, calcium lactate and zinc lactate.

21. The composition of claim 12, wherein said at least one lactic acid ingredient is an L-(+)-lactic acid salt selected from the group consisting of aluminum lactate, calcium lactate and zinc lactate.
